(19) United States
(12) Patent Application Publication
(10) Pub. No.: US 2011/0060136 A1
(43) Pub. Date: Mar. 10, 2011

(54) DENDRIMER-COATED MAGNETIC FINE PARTICLES, AND METHOD FOR PREPARING SAME AND UTILITY THEREOF

(75) Inventors: Tadashi MATSUNAGA, Tokyo (JP); Tsuyoshi TANAKA, Tokyo (JP); Keiichi HATAKEYAMA, Tokyo (JP); Takeo TANAAMI, Tokyo (JP); Hitoshi WAKE, Tokyo (JP); Tomoyuki TAGUCHI, Tokyo (JP); Takeyuki MOGI, Tokyo (JP)

(73) Assignees: TOKYO UNIVERSITY OF AGRICULTURE AND TECHNOLOGY, Tokyo (JP); YOKOGAWA ELECTRIC CORPORATION, Tokyo (JP)

(21) Appl. No.: 12/878,755

(22) Filed: Sep. 9, 2010

(30) Foreign Application Priority Data
Sep. 9, 2009 (JP) 2009-207802

(51) Int. Cl. C07H 1/06; H01F 1/00

(52) U.S. Cl. 536/23.1; 252/62.54

(57) ABSTRACT

Dendrimer-coated magnetic fine particles comprise magnetic fine particles, a lipid bilayer covering a surface of individual magnetic fine particles, and a dendrimer bound to an outer layer of the lipid bilayer. With the dendrimer-coated magnetic fine particles, the dendrimer is positively charged are brought into contact with a nucleic acid-containing solution to adsorb the nucleic acid on the dendrimer, while the nucleic acid-adsorbed fine particles are collected by magnetic force to recover the nucleic acid from the solution.

### BACKGROUND OF THE INVENTION

This invention relates to dendrimer-coated magnetic fine particles and also to a method for preparing the same and a utility thereof applied for recovery or purification of nucleic acids.

### TECHNICAL BACKGROUND

In the extraction method of nucleic acids hitherto employed from of old, it has been typical to use phenolic extraction making use of a toxic organic solvent such as phenol or chloroform. In recent years, there have been used, in place thereof, processes wherein a nucleic acid is adsorbed selectively on the surfaces of a silica carrier in the form of silica fine particles or silica membrane filter in a solution containing a high concentration of a chaotropic salt (guanidine hydrochloride, guanidine thiocyanate or the like) (see Vogelstein B., Gillespie D., Proc. Natl. Acad. Sci. USA, 1979, Vol. 76, p. 615-619). This principle enables a nucleic acid to be efficiently purified without use of such a dangerous solvent. Of the processes, the Boom process has been in wide use, in which silica-coated magnetic fine particles are used to permit a nucleic acid to be adsorbed and desorbed through chaotropic reaction (see Boom R., Sol C J., Salimans M M., Jansen C L., Wertheim-van Dillen P M., van der Noordaa J., J. Clinmicrobiol., 1990, Vol. 28, p 495-503). Moreover, there has been developed, as a technique based on a similar principle, a solid-phase reversible immobilization (SPRI) process which makes use of a phenomenon wherein a nucleic acid is bound selectively to magnetic fine particles modified with a carboxyl group in the presence of polyethylene glycol (PEG) (see Hawkins T L., O'Connor-Mortin T., Roy A., Santillan C., Nucleic Acids Res., 1994, Vol. 22, p. 4543-4544). These nucleic acid purification processes making use of magnetic fine particles do not need any operations of centrifugation, filtration, precipitation and the like, thus enabling a high-purity nucleic acid to be extracted and purified in a simple and rapid manner.

However, the Boom process essentially requires the use of irritative, toxic chaotropic salts under high concentration conditions in the nucleic acid adsorption step. Hence, the salt of high concentration is left even after through a washing step, with the possibility that this salt adversely influences subsequent reactions using enzymes, such as of genetic amplification, enzyme cleavage of DNA and the like. Moreover, in the operations of washing magnetic fine particles bound with a nucleic acid, 70% ethanol is employed. It has been pointed out that this ethanol likewise gives an adverse influence. Especially, where a nucleic acid should be handled at a very small reaction volume as with the case of microchip devices, high risk is involved in its incorporation. In the SPRI process, the adverse influences ascribed to the residue of a high concentration salt (NaCl) used in a nucleic acid adsorption step or the incorporation of ethanol in a washing step have become a problem as well.

To cope with these problems, there have been reported isolation techniques of nucleic acids, which make use of charge interaction between the solid phase surface for fixing a nucleic acid thereon and the nucleic acid (see International Laid-open Patent Publication No. 99/29703 and Japanese Laid-open Patent Publication No. 2004-521881 and Weidong Cao et al., Anal. Chem. 2006, Vol. 78, No. 20, P. 7222-7228). Moreover, the DNA extraction kit based on a principle (Charge-Switch technology) substantially same as the isolation technique has been commercially sold. These technologies are ones wherein a nucleic acid in a living body sample is brought into contact with an activated solid phase under certain pH conditions and a negatively charged nucleic acid is electrostatically bound to a positively charged polar group, such as chitosan, introduced at the solid phase surface. Subsequently, the pH of the solution is changed to switch the charge of the solid phase surface from positive to negative, thereby permitting the nucleic acid to be readily desorbed from the solid phase surface. These technologies are excellent in that since no chaotropic salt, high-concentration salt or ethanol is used, adverse influences on safety and also on reactions subsequent to nucleic acid extraction are lessened. Such purification techniques of nucleic acids making use of charges on magnetic fine particles have been expected as being applicable to microdevices. In application to inside microchannels, importance is placed on good dispersability and good magnetic responsiveness. The technique of satisfying them is set out in Yoza. B et al., J. Biosci. Bioeng. 2003, Vol. 95, No. 1, p. 21-26. More particularly, bacterial magnetic fine particles that have a single-domain structure and thus, are good at magnetic responsiveness although in nanosizes are provided as a core, and a polyamidoamide dendrimer is formed on the surfaces of the fine particles so as to permit the nucleic acid to be bound therewith. The dendritic structure of the dendrimer enables the surface amino group to be fixed at high density. Additionally, it has been elucidated that the fine particles are highly dispersible owing to the mutual surface charge repulsion thereof.

According to Yoza. B et al. (J. Biosci. Bioeng. 2003, Vol. 95, No. 1, p. 21-25), a polyamide dendrimer formed on the surfaces has two functions of nucleic acid adsorption and prevention of coagulation between a nucleic acid and the particles, and both are performed by the action of positive surface charge. Accordingly, nucleic acid adsorption leads to cancellation of the positive surface charge, so that mutual coagulation of the magnetic fine particles is caused. Although this coagulation is advantageous with ease in recovery of the magnetic fine particles, a disadvantage is also involved due to the poor efficiency in the steps of washing and desorbing a nucleic acid.

### SUMMARY OF THE INVENTION

It is therefore an object of the invention to provide magnetic fine particles that are coated with a dendrimer and are unlikely to cause coagulation after adsorption of an intended substance such as a nucleic acid and also a method for preparing same and a method for recovering or purifying a nucleic acid by use thereof.

We have made intensive studies and, as a result, found that if a lipid bilayer is interposed between the surface of magnetic fine particles and a dendrimer, an average distance between the magnetic fine particles after being bound with an intended substance such an a nucleic acid becomes large, whereby mutual coagulation between the magnetic fine particles is unlikely to occur. The invention has been accomplished based on this finding.

More particularly, the invention contemplates to provide dendrimer-coated magnetic fine particles comprising magnetic fine particles, a lipid bilayer covering the surface of individual magnetic particles, and a dendrimer bound to an outer layer of the lipid bilayer.

The invention also provides a method for preparing dendrimer-coated magnetic fine particles, which method comprising the steps of

(1) providing magnetic fine particles having a functional group on a surface thereof;

(1) providing magnetic fine particles having a functional group on a surface thereof;

(2) reacting a first amphipathic lipid, which has a hydrophobic moiety and a hydrophilic moiety having a functional group capable of reacting and binding with the first-mentioned functional group on said magnetic fine particles, with said first-mentioned functional group to bind said first amphipathic lipid to the surface of said fine particles in such a way that the hydrophobic moiety is turned outside;

(3) bringing a second amphipathic lipid, which has a hydrophobic moiety and a hydrophilic moiety having a functional group capable of reacting and binding with a functional group present at a base end portion of a dendrimer, into contact with the fine particles after the step (2) in an aqueous medium to form an outer layer of the lipid bilayer by self-assembly; and

(4) reacting the fine particles after the step (3) with said dendrimer to bind the functional group present at the base end portion of said dendrimer with the functional group of said second amphipathic lipid.

Further, the invention also provides a method for preparing dendrimer-coated magnetic fine particles, which method comprising the steps of;

(1) providing magnetic fine particles having a functional group on a surface thereof;

(2) reacting a first amphipathic lipid, which has a hydrophobic moiety and a hydrophilic moiety having a functional group capable of reacting and binding with the first-mentioned functional group on said magnetic fine particles, with said first-mentioned functional group to bind said first amphipathic lipid to the surface of said magnetic fine particles in such a way that the hydrophobic moiety is turned outside

(3) reacting a second amphipathic lipid, which has a hydrophobic moiety and a hydrophilic moiety having a functional group capable of reacting and binding with a functional group present at a base end portion of a dendrimer, with said dendrimer to bind the functional group present at the base end portion of said dendrimer with the functional group of said second amphipathic lipid; and

(4) bringing a dendrimer-bound lipid obtained in the step (3) into contact with the fine particles after the step (2) in an aqueous medium to form an outer layer of said lipid bilayer bound with said dendrimer by self-assembly.

The invention further provides a method for recovering a nucleic acid from a nucleic acid-containing solution, which method comprising the steps of:

(1) bringing dendrimer-coated magnetic fine particles whose dendrimer is positively charged into contact with a nucleic acid-containing solution to permit the nucleic acid to be adsorbed on the dendrimer; and

(2) collecting the fine particles adsorbed with nucleic acid by magnetic force.

After the recovery of the nucleic acid according to the above method, the nucleic acid is preferably desorbed from the fine particles thereby purifying the nucleic acid.

The dendrimer-coated magnetic fine particles of the invention have the lipid bilayer between the surface of individual magnetic fine particles and the dendrimer. When a nucleic acid or the like is recovered by use of the fine particles, an average distance between adjacent magnetic fine particles becomes larger than with the case using known magnetic fine particles having no lipid bilayer therein. This leads to the unlikelihood of causing coagulation of the particles. Accordingly, the steps of washing the magnetic fine particles after binding an intended substance thereto and the desorption step of the intended substance can be performed in an efficient manner. Especially, in case where an intended substance is recovered or purified within a microdevice, the unlikelihood of causing coagulation of the magnetic fine particles is beneficial. In the preparation methods of the invention, a thick lipid bilayer is formed by self-assembly, so that large-sized dendrimer-coated magnetic fine particles can be simply prepared.

### BRIEF DESCRIPTION OF THE DRAWINGS

FIG. 1 is a schematic view showing a reaction scheme of one example of a method for preparing dendrimer-coated magnetic fine particles adopted in an example of the invention; and

FIG. 2 is a graph showing a particle size and a size distribution, measured by means of a laser zeta potentiometer, of dendrimer-coated magnetic fine particles prepared in an example of the invention while comparing with known fine particles.

### EMBODIMENTS FOR CARRYING OUT THE INVENTION

As stated above, the dendrimer-coated magnetic fine particles of the invention comprise magnetic fine particles, a lipid bilayer covering the fine particles individually on the surface thereof, and a dendrimer bound to an outer layer of the lipid bilayer.

The magnetic fine particles are not critical in type so far as they are those particles, which are capable of being collected by magnetic force and become magnetized and which are able to impart a functional group capable of covalent bond with an amphipathic lipid described hereinafter. Mention is made of magnetic bacteria-derived magnetic fine particles, metal or plastic magnetic fine particles, magnetic beads and the like. The diameter of the magnetic fine particles are not critical and is preferably at about 50 to 100 nm. Of these, magnetic bacteria-derived magnetic fine particles are preferred because they have a single-domain structure and thus are good at magnetic responsiveness although in nanosizes. It is known that the magnetic bacteria have a magnetosome that consists of a sequence of ten to twenty magnetite fine particles having a diameter of about 50 to 100 nm in the bacterial body. The magnetite fine particles can be favorably used in the practice of the invention. The magnetic bacteria known in the art include Magnetospirillum magneticums AMB-1 and MGT-1, Mangetospirillum gryphiswaldense MSR-1, Aquaspirillium magnetotacticum MS-1 and the like. It will be noted that a method for recovering and purifying a nucleic acid by use of an amino group-bearing dendrimer (which will be described hereinafter) using magnetic bacteria-derived magnetic fine particles as a fixation carrier has been already found by us and is now known in the art (see, for example, Japanese Laid-open Patent Publication No. 2009-65849).

The surface of each magnetic fine particle is covered with a lipid bilayer. The lipid bilayer is one wherein an amphipathic lipid having a hydrophobic moiety and a hydrophilic moiety in one molecule is constituted of two layers in an aqueous medium in such a way that the hydrophilic moiety is turned outside. The lipid bilayer itself is well known as being a main constituent element of a living membrane. It is also well known that the amphipathic lipid making up the lipid bilayer not only serves as a living membrane, but also is a constituent element of a liposome widely used such as in drug delivery systems.

The lipid bilayer used in the practice of the invention is not critical so far as it is formed of an amphipathic lipid capable of performing self-assembly or self-organization (wherein a bilayer is automatically formed merely upon mixing) in an aqueous medium. It is preferred to use a bilayer that is fundamentally constituted of a known amphipathic lipid forming a living membrane or the like. For such an amphipathic lipid, phospholipids having phosphoric esters as a hydrophilic moiety are preferred, of which a glycerophospholipids having a phosphatidyl group such as phosphatidylethanolamine are more preferred. Although the most preferred hydrophilic moiety includes a long-chain alkyl group (having generally about 12 to 30 carbon atoms, preferably about 15 to 24 carbon atoms), the long-chain alkyl group may be substituted with other type of substituent within a range not impeding the self assembly of the lipid bilayer. In the case of the glycerophospholipids, the number of the long-chain alkyl group is preferably to be 2 in one molecule.

It is preferred from the standpoints of the stability of the particulate structure and the preparation efficiency that the inner layer (i.e. a layer at the side of the magnetic fine particles) of the lipid bilayer is bound to the surface of the magnetic fine particles through covalent bond and the outer layer (i.e. a layer at a side binding with a dendrimer described hereinafter) of the lipid bilayer is covalently bound to the dendrimer. Hence, the amphipathic lipid acting as the lipid bilayer is made substantially of one set out hereinabove and has preferably functional groups capable of chemical binding with other functional groups, respectively. This will be described in more detail in the illustration of a preparation method described hereinafter.

The lipid bilayer is bound to a dendrimer at the outer layer thereof. The dendrimer means a dendritic polymer and has been extensively studied since when a desired type of functional group is introduced into the polymer, there are imparted thereto such excellent properties that the number of the desired functional groups capable of being fixed per unit area of a carrier can be remarkably increased. As will be described hereinafter, in order to recover or purify a nucleic acid by use of the fine particles of the invention, it is preferred that the dendrimer is positively charged and has an amino group. In this sense, a poly(amidoamine) (PAMAM) dendrimer is especially preferred.

The PAMAM dendrimer per se is known (e.g. in Yoza. B. et al., J. Biosci. Bioeng. 2003, Vol. 95, No. 1, p. 21-26) and is generally made of a branched structure of an alkylamine (wherein part of the carbon atoms may be substituted with a sulfur atom, like cystamine) core (generally having about 2 to 12 carbon atoms) and a tertiary amine. Commercially available PAMA dendrimers include those of a variety of generations (wherein the generation means one as corresponding to what number branch from a core and is controlled by the number of reaction cycles for branch growth) using different types of cores. In the practice of the invention, such commercially available dendrimers can be favorably used. We have already proposed dendrimer-fixed magnetic fine particles wherein PAMAM dendrimer is fixed on the surface of magnetic fine particle and also a method for extracting a nucleic acid or protein by use of the particles, and filed for an application (see Japanese Laid-open Patent Publication No. 2004-150797). With the PAMAM dendrimer, it is known that the number of amino groups, present at a branch terminal, per unit area becomes maximum for the sixth generation. In this sense, it is most preferred to use a dendrimer of the sixth generation although other generations of dendrimers may also be usable.

The dendrimer is preferably bound to the outer layer of the lipid bilayer through covalent bond. Accordingly, the core of the dendrimer should preferably consist of one that contains an S—S bond such as of cystamine (wherein the carbon atoms at the 4 and 5 positions of 1,6-hexanediamine are replaced by sulfur atom) or the like. In this case, an advantage is such that a thiol group generated by cutting off the S—S bond can be used for binding to the hydrophilic moiety of an amphipathic lipid. It will be noted that the sixth generation PAMAM dendrimer using cystamine as a core is commercially available and this commercial product can be preferably used in the practice of the invention.

Next, the method for preparing the dendrimer-coated magnetic fine particles of the invention is now described.

Initially, magnetic fine particles having a functional group on the surface of individual particles are provided. This step per se is known in the art and is described, for example, in Japanese Laid-open Patent Publication No. 2006-280277. The magnetic fine particles are just as stated hereinbefore, and magnetic bacteria-derived magnetic fine particles are preferred. The magnetic bacteria-derived magnetic fine particles have a bacteria-derived lipid bilayer on the surface thereof, and a difficulty is involved in subjecting a dendrimer to covalent bond therewith. Hence, it is convenient to eliminate the bacteria-derived lipid bilayer by the action of a surface active agent such as 1% sodium dodecylsulfate (SDS), an organic solvent, a strong alkali or the like.

The functional group on the surface of the magnetic fine particles may be one capable of binding with a substituent group at the hydrophilic moiety of a first amphipathic lipid and especially an amino group is preferred. The impartment of an amino group to magnetic bacteria-derived magnetic fine particles can be performed by subjecting the surface of the fine particles to amino silane treatment such as with a known amino silane coupling agent or an aminosilylation agent. Preferred examples of the amino silane coupling agent include amino group-containing silane derivatives such as 3-[2-(2-aminoethyl)-ethylamino]propyltrimethoxysilane (AEEA) and the like. When the surface of the particles is subjected to amino silane treatment with the above-mentioned amino silane coupling agent, it is preferred to permit the hydroxyl group present in the particles to be exposed to the surface. For instance, when magnetic bacteria-derived magnetic fine particles are adopted as the particles, the amino silane treatment of the surface thereof is carried out such that the bacteria-derived lipid bilayer existing on the particle surface is removed to activate the surface hydroxyl group, thereby enabling the aminosilylation reaction or amino silane coupling reaction to be promoted. One example of more specific reaction conditions is described in an example described hereinafter.

In a subsequent second step, a first amphipathic liquid, which has a hydrophobic moiety and a hydrophilic moiety having a functional group capable of reacting and binding with the functional group, preferably an amino group, of the fine particles, is reacted with the functional group on the magnetic fine particles, so that the first amphipathic liquid is bound to the surface of the magnetic fine particles so as to allow the hydrophobic moiety to be turned outside. Preferred examples of the function group present at the hydrophilic moiety of the first amphipathic lipid and capable of binding with the amino group on the surface of the magnetic fine particles include a hydrosuccinimidyl (NHS) ester group, a sulfohydroxylsuccinimidyl (sulfo-NHS) ester group, an imidoester group, an aldehyde group, an isothiocyanate group and the like although not limited thereto. A variety of glycerophospholipids having an NHS ester group, which are favorably used in the invention and include distearoyl N-(succinimidyl-glutaryl)-L-a-phosphatidylethanolamine (DSPE-NHS) or the like wherein hydrosuccinimidyl (NHS) ester group is added to a hydrophilic moiety of a glycerophospholipid having a phosphatidyl group such as phosphatidylethanolamine, are commercially sold, and these commercial products can be favorably used.

The reaction between the magnetic fine particles having an amino group and the phospholipid having an NHS group can be carried out in an organic solvent such as, for example, DMSO generally at a temperature of 10° C. to 40° C., preferably at room temperature generally for a reaction time of about 15 to 60 minutes, preferably about 20 to 40 minutes. In order to prevent the magnetic fine particles from being coagulated, the reaction solution should preferably be subjected to ultrasonic dispersion treatment. The concentration of the magnetic fine particles upon the reaction is generally at about 0.2 mg/ml to 1.0 mg/ml, preferably at about 0.4 mg/ml to 0.6 m/ml. The concentration of the phospholipid having an NHS ester group is generally at about 0.5 mM to 2 mM, preferably at about 0.8 mM to 1.2 mM.

In a subsequent third step, a second amphipathic liquid having a hydrophobic moiety and a hydrophilic moiety having a functional group capable of reacting and binding with a functional group present at a base end portion (i.e. a basal portion more than a first branch of a dendrimer) of a dendrimer is brought into contact with the fine particles after the second step in the aqueous medium thereby forming an outer layer of the lipid bilayer by self-assembly. The second amphipathic liquid is fundamentally just as stated hereinbefore and the hydrophilic moiety thereof should be one having a functional group capable of binding with the dendrimer. The dendrimer used is preferably a PAMAM dendrimer making use, as a core, of cystamine having an S—S bond or the like. In this case, because the SH group of the dendrimer is released, those having a functional group, such as a maleimido group, which is able to react with the SH group, are preferred. A variety of glycerophospholipids having a maleimido group, such as distearoyl N-(3-maleimido-1-oxypropyl)-L-a-phosphatidylethanolamine wherein a maleimido group is added to a hydrophilic moiety of a glycerophospholipid having a phosphatidyl group such as a phosphatidylethanolamine, are commercially available and these commercial products can be favorably used.

The self-assembly reaction in the third step can be carried out by mixing the magnetic fine particles, which have been bound with an inner layer of the lipid bilayer in the second step, with the second amphipathic liquid in an aqueous buffer solution such as a phosphate buffer solution (PBS) under heating conditions and pouring the resulting mixture into an aqueous buffer solution such as PBS at temperatures lower than that of the mixture, preferably at room temperature. The concentration of the magnetic fine articles in the mixing step under heating conditions is generally at about 0.2 mg/ml to 1.0 mg/ml, preferably at about 0.4 to 0.6 mg/ml. The concentration of the phospholipid having a maleimido group is generally at about 0.5 mM to 2 mM, preferably about 0.8 mM to 1.2 mM. The mixing time is generally 2 minutes to 10 minutes, preferably at about 4 to 6 minutes. During the mixing step under heating conditions, the reaction solution should preferably be subjected to ultrasonic dispersion treatment. The aqueous medium to be poured thereinto should preferably be used in excess over the heated mixture and is used generally about eighthold to twelvefold on the volume basis. After the pouring, the reaction solution is allowed to stand generally for about 30 minutes to 60 minutes, preferably for about 40 minutes to 50 minutes, during which the inner layer consisting of the first amphipathic liquid bound on the magnetic fine particles and the outer layer consisting of the second amphipathic liquid are built up in such a way that mutual hydrophobic moieties come into contact with each other by the hydrophobic interaction to be laminated thereby forming a lipid bilayer.

In a subsequent fourth step, the fine particles obtained after the above third step and the dendrimer are reacted with each other to bind the functional group present at the base end portion of the dendrimer with the functional group of the second amphipathic liquid. As stated hereinabove, the dendrimer used in the invention preferably consists of a PAMAM dendrimer having an S—S bond as a core. When such a dendrimer is treated with a reducing agent such as dithiothreitol, the S—S bond of the core is cut off to obtain a dendrimer having a free SH group at the base end portion. The resulting free SH group is bound to a functional group, preferably a maleimido group, present at the hydrophilic moiety of the second amphipathic lipid. The reaction between the maleimido group and the SH group can be carried out by mixing the magnetic fine particles and the dendrimer in an aqueous buffer solution, such as PBS, preferably while dispersing under ultrasonic treatment. The reaction temperature is generally at 10° C. to 40° C., preferably at room temperature. The reaction time is generally 30 minutes to 2 hours, preferably at about 40 minutes to 80 minutes. The concentration of the dendrimer upon the reaction is generally at about 0.2 mg/ml to 1.0 mg/ml, preferably at about 0.4 mg/ml to 0.6 mg/ml. The concentration of the dendrimer is generally at 0.001 mM to 0.02 mM, preferably at about 0.005 mM to 0.015 mM. The fourth self-assembly step can be carried out under similar conditions as in the third self-assembly step in the above-stated preparation method.

With the fourth step, the dendrimer is bound on the outer layer of the lipid bilayer, thereby obtaining the dendrimer-coated magnetic fine particles of the invention. It is preferable that the magnetic fine particles thus obtained is used after washing in an aqueous buffer solution such as PBS.

In the preparation methods of the invention, although the outer layer of the lipid bilayer is laminated in the third step, and the dendrimer is bound on the outer layer of the lipid bilayer in the fourth step, the dendrimer may be bound on the second amphipathic lipid forming the outer layer in the third step while a bound material of the second amphipathic lipid and the dendrimer is subjected to reaction in the final fourth step, thereby forming the lipid bilayer by self-assembly. In this case, the reaction between the second amphipathic lipid and the dendrimer can be carried out at a temperature of 10° C. to 40° C., preferably at room temperature. A reaction time is generally about 60 minutes to 2 hours, preferably about 40 to 80 minutes. The concentration of the second amphipathic lipid upon the reaction is generally at about 0.5 mM to 2.0 mM, preferably at about 0.8 mM to 1.2 mM and the concentration of the dendrimer is generally at about 0.001 mM to 0.02 mM, preferably at about 0.005 mM to 0.015 mM. Further, the self-assembly step in the fourth step can be carried out under the same conditions as that in the third step of the above-stated preparation methods.

The dendrimer-coated magnetic fine particles of the invention can be used for recovery or purification of a nucleic acid or protein substantially in the same way as the known dendrimer-coated magnetic fine particles set forth in Japanese Laid-open Patent Publication Nos. 2004-150797 and 2009-65849. Where the dendrimer is positively charged in water preferably as having an amino group or the like, a nucleic acid such as DNA or RNA, which is negatively charged in water, can be adsorbed on the magnetic fine particles by the electrostatic interaction therebetween. More particularly, the magnetic fine particles of the invention is brought into contact with a nucleic acid-containing solution to adsorb the nucleic acid on the dendrimer, followed by collecting the nucleic acid-adsorbed fine particles by magnetic force to recover the nucleic acid from the solution. The nucleic acid-containing solutions include, for example, any of solutions containing materials from various types of organisms such as cultured cells, animal-derived cells or tissues (blood, serum, buffy coat, body fluid, lymphocyte and the like), plant-derived cells or tissues, or bacteria, fungi, viruses and the like. The amount of the magnetic fine particles to be brought into contact with the nucleic acid-containing solution may be appropriately determined depending on the concentration of an expected nucleic acid and the amount of the nucleic acid intended for recovery and is generally at about 0.1 mg/ml to 1.0 mg/ml. The adsorption reaction may be at room temperature and reaction time is generally about 30 seconds to 5 minutes. The magnetic fine particles may be located in microchannels of a microdevice to adsorb a nucleic acid.

The nucleic acid-adsorbed magnetic fine particles can be collected by magnetic force in a usual manner.

The nucleic acid adsorbed on the collected magnetic fine particles can be purified by desorption thereof from the particles. The manner of desorption is known in the art as set forth, for example, in Japanese Laid-open Patent Publication Nos. 2004-150797 and 2009-65849 and can be carried out by a thermal treatment, a surfactant treatment or a treatment with a desorbing agent containing a phosphoric group. The thermal treatment conditions generally include about 70° C. to 90° C. and about 10 minutes to 30 minutes. The surfactant used includes sodium dodecylsulfate, Triton X-100 (commercial name), Tween 20 (commercial name) or the like and the concentration thereof is generally at about 001 wt % to 1 wt %. For the desorbing agent containing a phosphoric group, there can be used a deoxyribonucleoside diphosphate such as ADP or the like, and a deoxyribonucleoside triphosphate such as ATP or the like. The concentration upon use is generally at about 1.0 mM to 500 mM and coexistence of a low-concentration organic solvent such as ethanol is preferred.

The nucleic acid desorbed from the magnetic fine particles can be used for an intended purpose and can, of course, be amplified after subjecting to a nucleic acid amplification technique such as PCR or the like. In this case, it is possible to carry out the above-sated desorption step in a PCR reaction solution for performing a nucleic acid amplification method in the presence of the magnetic fine particles wherein the nucleic acid has been desorbed. In this way, the case where the desorption is made at a point of use of the nucleic acid is within a category of the purification method of the invention.

### EXAMPLES

The invention is more particularly described by way of examples, which should not be construed as limiting the invention thereto.

### Example 1

### Preparation of Magnetic Fine Particles

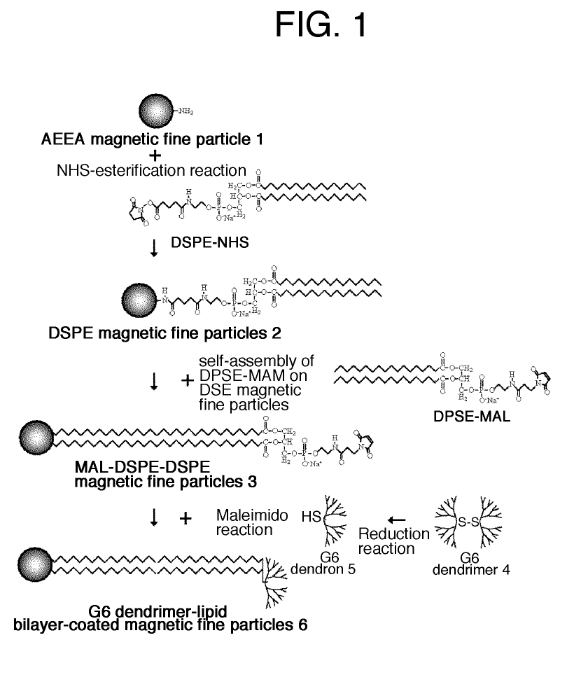

According to the reaction scheme shown in FIG. 1, dendrimer-coated magnetic fine particles of the invention were prepared. It will be noted that a hydrophobic moiety of a first amphipathic lipid of “MAL-DSPE-DSPE magnetic fine particles 3” and “G6 dendrimer-lipid bilayer coated magnetic fine particles 6” in FIG. 1 is omitted in the figure for simplicity.

Initially, magnetic fine particles modified with a lipid on the surface thereof were prepared. Magnetic bacteria (Magnetosirillum magneticum AMB-1) were isolated and prepared according to a known procedure, after which a lipid bilayer was removed from the surface of the magnetic fine particles (average particle size: 80 nm) (see Biotechnology and Bioengineering, Volume 94, Issue 5, pages 862-868 (2006)). The lipid bilayer was removed with a 1% SDS solution. After washing three times with distilled water, 20 ml of an ammonium peroxide solution (H₂O:H₂O₂:NH₃=5:1:1) was added to and dispersed by ultrasonic waves, followed by allowing to stand for 10 minutes thereby activating the hydroxyl group on the surfaces of the magnetic fine particles. After washing three times with anhydrous methanol, the resulting magnetic fine particles were reacted with AEEA for 10 minutes while subjecting an ethanol solution of 2% AEEA to ultrasonic dispersion. After the reaction, the magnetic fine particles were washed three times with methanol. After further washing once with DMF, the particles were treated in DMF at 120° C. for 30 minutes for stabilization of the silane coupling, thereby preparing AEEA magnetic fine particles 1.

As a first amphipathic liquid, there was used distearoyl N-(succinimidyl-glutaryl)-L-a-phosphatidylethanolamine (DSPE-NHS, commercial product) having a hydrosuccinimidyl (NHS) ester group reactive with an amino group present on the surface of the AEEA magnetic fine particles. A DSPE-NHS solution adjusted to 1 mM by means of DMSO was added so as to make a concentration of the AEEA magnetic fine particles at 0.5 mg/ml and heated to 65° C. while subjecting to ultrasonic dispersion, followed by pouring into 10 ml of PBS (under ultrasonic dispersion at a pH of 7.4 at room temperature). MAL-DSPE-DSPE-modified magnetic fine particles 3 were prepared by hydrophobic interaction wherein DSPE-MAL was self-assembled relative to the SDPSE on the particles.

Next, a dendron bound to the fine particle was prepared. 400 μA of DTT adjusted to 0.5 mM by means of PBS was added to 100 μl of a methanol solution of a 0.5 mM G6 dendrimer (PAMAM dendrimer, cystamine core, sixth generation). Thereafter, while agitating, the mixture was incubated at room temperature for 12 hours to reduce the cystamine core thereby providing G6 dendron 5. The cleavage of the cystamine core permitted the thiol group to be in a reactive condition.

A G6 dendron solution prepared by use of PBS was added to the MAL-DSPE-DSPE-modified magnetic fine particles, followed by ultrasonic dispersion at room temperature for 60 minutes. The dendron was modified through the lipid bilayer by the reaction between the maleimido group on the particles and the thiol of the dendron. After washing with PBS, collected particles were provided as G6 dendrimer-lipid coated magnetic particles 6.

### Example 2

Transmission electron microscope images of the magnetic fine particles prepared in Example 1 and dendrimer-coated magnetic fine particles (Japanese Laid-open Patent Publication No. 2009-65849) similar to those of Example 1 except that no lipid bilayer was formed were taken. As a result, it was found that the structure formed by the lipid bilayer was confirmed in the magnetic fine particles prepared according to the method of Example 1. The thicknesses of the respective particles was measured from the TEM images, revealing that the particle A that was free of the lipid bilayer was at about 6.5 nm in thickness and the particle B having the lipid bilayer was at about 11 nm in thickness. Where the G6 dendron was regarded as a hemisphere, the height was at 3.35 nm (Tomalia et al. 2003), the molecular length of GMBS used as a crosslinking agent in the particle A was at 0.73 nm (Thermo Scientific), and the thickness of the lipid bilayer was at about 5 to 10 nm. From the above, it was suggested that the G6 dendrimer liquid bilayer-coated magnetic particles were prepared as expected.

The capabilities of recovery and desorption of DNA were evaluated. More particularly, the following procedure was carried out. A 1DNA solution adjusted to 100 ng/40 μl by use of a 10 mM Tris-HCl buffer solution (pH 7.5) was added to 10 μg of the prepared particles and after ultrasonic dispersion, the mixture was allowed to stand for 1 minute to allow the 1DNA to be adsorbed on the particles. The amount of 1DNA contained in a supernatant liquid obtained after centrifugal recovery (20400 g, 5 minutes) of the particles was quantitatively determined by use of Picogreen that was an intercalator, from which the amount of 1DNA adsorbed on the particles was calculated. Next, the particles adsorbed with 1DNA were washed three times with a 10 mM Tris-HCl buffer solution, after which 40 μl of a 1 M phosphate buffer solution (pH 7.0) was added and then subjected to ultrasonic dispersion, followed by allowing to stand in a thermostatic chamber at 80° C. for 20 minutes to desorb 1DNA from the particles. After centrifugal recovery (20400 g, 5 minutes) of the particles, the amount of 1DNA in the resulting supernatant liquid was quantitatively determined by use of Picogreen to calculate the amount of 1DNA desorbed from the particles. As a result, about 150 ng of 1DNA could be recovered by use of 10 μg of the dendrimer-coated magnetic fine particles. The recovery rate (amount of desorbed 1DNA/amount of adsorbed 1DNA) was at about 96%.

As will be apparent from the above results, the capabilities of recovery and desorption of DNA in case where the magnetic fine particles of the invention were used were substantially equal to those of the known magnetic fine particles, and there was found no lowering of the capabilities of recovery and desorption as would be caused by the formation of the lipid bilayer.

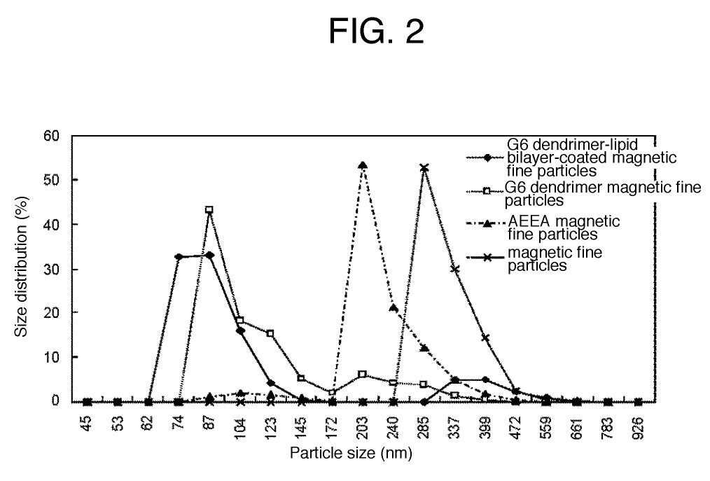

Further, in order to check the dispersability of the fine particles, the size distribution of the respective types of fine particles after ultrasonic dispersion was compared by use of a laser zeta potentiometer. The results are shown in FIG. 2.

As shown in FIG. 2, the dendrimer-coated magnetic fine particles of the invention wherein the lipid bilayer was formed were smaller in apparent particle size than the known dendrimer-coated magnetic fine particles wherein no liquid bilayer was formed, revealing that the dispersability of the particles of the invention was better.

1. Dendrimer-coated magnetic fine particles comprising magnetic fine particles, a lipid bilayer covering a surface of individual magnetic fine particles, and a dendrimer bound to an outer layer of said lipid bilayer.

2. The fine particles according to claim 1, wherein said dendrimer is positively charged.

3. The fine particles according to claim 2, wherein said dendrimer has an amino group.

4. The fine particles according to claim 1, wherein an inner layer of said lipid bilayer is covalently bound to the surface of said magnetic fine particles, and an outer layer of said lipid bilayer is covalently bound to said dendrimer.

5. A method for preparing dendrimer-coated magnetic fine particles defined in claim 1, the method comprising the steps of:
(1) providing magnetic fine particles having a functional group on a surface thereof;
(2) reacting a first amphipathic lipid, which has a hydrophobic moiety and a hydrophilic moiety having a functional group capable of reacting and binding with the first-mentioned functional group on said magnetic fine particles, with said first-mentioned functional group to bind said first amphipathic lipid to the surface of said fine particles in such a way that the hydrophobic moiety is turned outside;
(3) bringing a second amphipathic lipid, which has a hydrophobic moiety and a hydrophilic moiety having a functional group capable of reacting and binding with a functional group present at a base end portion of a dendrimer, into contact with the fine particles after the step (2) in an aqueous medium to form an outer layer of the lipid bilayer by self-assembly; and
(4) reacting the fine particles after the step (3) with said dendrimer to bind the functional group present at the base end portion of said dendrimer with the functional group of said second amphipathic lipid.

6. A method for preparing dendrimer-coated magnetic fine particles defined in claim 1, the method comprising the steps of:
(1) providing magnetic fine particles having a functional group on a surface thereof;
(2) reacting a first amphipathic lipid, which has a hydrophobic moiety and a hydrophilic moiety having a functional group capable of reacting and binding with the first-mentioned functional group on said magnetic fine particles, with said first-mentioned functional group to bind said first amphipathic lipid to the surface of said magnetic fine particles in such a way that the hydrophobic moiety is turned outside
(3) reacting a second amphipathic lipid, which has a hydrophobic moiety and a hydrophilic moiety having a functional group capable of reacting and binding with a functional group present at a base end portion of a dendrimer, with said dendrimer to bind the functional group present at the base end portion of said dendrimer with the functional group of said second amphipathic lipid; and
(4) bringing a dendrimer-bound lipid obtained in the step (3) into contact with the fine particles after the step (2) in an aqueous medium to form an outer layer of said lipid bilayer bound with said dendrimer by self-assembly.

7. A method for recovering a nucleic acid in a solution, method comprising the steps of:
(1) bringing dendrimer-coated magnetic fine particles defined, in claim 1 in which said dendrimer is positively charged, into contact with a nucleic acid-containing solution to adsorb said nucleic acid on said dendrimer; and
(2) collecting the fine particles adsorbed with the nucleic acid by magnetic force.

8. A method for purifying a nucleic acid in a solution comprising the steps of recovering a nucleic acid according to the method defined in claim 7, and desorbing said nucleic acid from the fine particles.

9. A method for preparing dendrimer-coated magnetic fine particles defined in claim 2, the method comprising the steps of:
(1) providing magnetic fine particles having a functional group on a surface thereof;
(2) reacting a first amphipathic lipid, which has a hydrophobic moiety and a hydrophilic moiety having a functional group capable of reacting and binding with the first-mentioned functional group on said magnetic fine particles, with said first-mentioned functional group to bind said first amphipathic lipid to the surface of said fine particles in such a way that the hydrophobic moiety is turned outside;
(3) bringing a second amphipathic lipid, which has a hydrophobic moiety and a hydrophilic moiety having a functional group capable of reacting and binding with a functional group present at a base end portion of a dendrimer, into contact with the fine particles after the step (2) in an aqueous medium to form an outer layer of the lipid bilayer by self-assembly; and
(4) reacting the fine particles after the step (3) with said dendrimer to bind the functional group present at the base end portion of said dendrimer with the functional group of said second amphipathic lipid.

10. A method for preparing dendrimer-coated magnetic fine particles defined in claim 3, the method comprising the steps of:
(1) providing magnetic fine particles having a functional group on a surface thereof;
(2) reacting a first amphipathic lipid, which has a hydrophobic moiety and a hydrophilic moiety having a functional group capable of reacting and binding with the first-mentioned functional group on said magnetic fine particles, with said first-mentioned functional group to bind said first amphipathic lipid to the surface of said fine particles in such a way that the hydrophobic moiety is turned outside;
(3) bringing a second amphipathic lipid, which has a hydrophobic moiety and a hydrophilic moiety having a functional group capable of reacting and binding with a functional group present at a base end portion of a dendrimer, into contact with the fine particles after the step (2) in an aqueous medium to form an outer layer of the lipid bilayer by self-assembly; and
(4) reacting the fine particles after the step (3) with said dendrimer to bind the functional group present at the base end portion of said dendrimer with the functional group of said second amphipathic lipid.

11. A method for preparing dendrimer-coated magnetic fine particles defined in claim 4, the method comprising the steps of:
(1) providing magnetic fine particles having a functional group on a surface thereof;
(2) reacting a first amphipathic lipid, which has a hydrophobic moiety and a hydrophilic moiety having a functional group capable of reacting and binding with the first-mentioned functional group on said magnetic fine particles, with said first-mentioned functional group to bind said first amphipathic lipid to the surface of said fine particles in such a way that the hydrophobic moiety is turned outside;
(3) bringing a second amphipathic lipid, which has a hydrophobic moiety and a hydrophilic moiety having a functional group capable of reacting and binding with a functional group present at a base end portion of a dendrimer, into contact with the fine particles after the step (2) in an aqueous medium to form an outer layer of the lipid bilayer by self-assembly; and
(4) reacting the fine particles after the step (3) with said dendrimer to bind the functional group present at the base end portion of said dendrimer with the functional group of said second amphipathic lipid.

12. A method for preparing dendrimer-coated magnetic fine particles defined in claim 2, the method comprising the steps of:
(1) providing magnetic fine particles having a functional group on a surface thereof;
(2) reacting a first amphipathic lipid, which has a hydrophobic moiety and a hydrophilic moiety having a functional group capable of reacting and binding with the first-mentioned functional group on said magnetic fine particles, with said first-mentioned functional group to bind said first amphipathic lipid to the surface of said magnetic fine particles in such a way that the hydrophobic moiety is turned outside
(3) reacting a second amphipathic lipid, which has a hydrophobic moiety and a hydrophilic moiety having a functional group capable of reacting and binding with a functional group present at a base end portion of a dendrimer, with said dendrimer to bind the functional group present at the base end portion of said dendrimer with the functional group of said second amphipathic lipid; and
(4) bringing a dendrimer-bound lipid obtained in the step (3) into contact with the fine particles after the step (2) in an aqueous medium to form an outer layer of said lipid bilayer bound with said dendrimer by self-assembly.

13. A method for preparing dendrimer-coated magnetic fine particles defined in claim 3, the method comprising the steps of:
(1) providing magnetic fine particles having a functional group on a surface thereof;
(2) reacting a first amphipathic lipid, which has a hydrophobic moiety and a hydrophilic moiety having a functional group capable of reacting and binding with the first-mentioned functional group on said magnetic fine particles, with said first-mentioned functional group to bind said first amphipathic lipid to the surface of said magnetic fine particles in such a way that the hydrophobic moiety is turned outside
(3) reacting a second amphipathic lipid, which has a hydrophobic moiety and a hydrophilic moiety having a functional group capable of reacting and binding with a functional group present at a base end portion of a dendrimer, with said dendrimer to bind the functional group present at the base end portion of said dendrimer with the functional group of said second amphipathic lipid; and
(4) bringing a dendrimer-bound lipid obtained in the step (3) into contact with the fine particles after the step (2) in an aqueous medium to form an outer layer of said lipid bilayer bound with said dendrimer by self-assembly.

14. A method for preparing dendrimer-coated magnetic fine particles defined in claim 4, the method comprising the steps of:
(1) providing magnetic fine particles having a functional group on a surface thereof;
(2) reacting a first amphipathic lipid, which has a hydrophobic moiety and a hydrophilic moiety having a functional group capable of reacting and binding with the first-mentioned functional group on said magnetic fine particles, with said first-mentioned functional group to bind said first amphipathic lipid to the surface of said magnetic fine particles in such a way that the hydrophobic moiety is turned outside
(3) reacting a second amphipathic lipid, which has a hydrophobic moiety and a hydrophilic moiety having a functional group capable of reacting and binding with a functional group present at a base end portion of a dendrimer, with said dendrimer to bind the functional group present at the base end portion of said dendrimer with the functional group of said second amphipathic lipid; and
(4) bringing a dendrimer-bound lipid obtained in the step (3) into contact with the fine particles after the step (2) in an aqueous medium to form an outer layer of said lipid bilayer bound with said dendrimer by self-assembly.

15. A method for recovering a nucleic acid in a solution, method comprising the steps of:
(1) bringing dendrimer-coated magnetic fine particles defined in claim 2, in which said dendrimer is positively charged, into contact with a nucleic acid-containing solution to adsorb said nucleic acid on said dendrimer; and
(2) collecting the fine particles adsorbed with the nucleic acid by magnetic force.

16. A method for recovering a nucleic acid in a solution, method comprising the steps of:
(1) bringing dendrimer-coated magnetic fine particles defined in claim 3, in which said dendrimer is positively charged, into contact with a nucleic acid-containing solution to adsorb said nucleic acid on said dendrimer; and
(2) collecting the fine particles adsorbed with the nucleic acid by magnetic force.

17. A method for recovering a nucleic acid in a solution, method comprising the steps of:
(1) bringing dendrimer-coated magnetic fine particles defined in claim 4, in which said dendrimer is positively charged, into contact with a nucleic acid-containing solution to adsorb said nucleic acid on said dendrimer; and
(2) collecting the fine particles adsorbed with the nucleic acid by magnetic force.
